(19) United States
(12) Patent Application Publication
(10) Pub. No.: US 2018/0139998 A1
(43) Pub. Date: May 24, 2018

(54) LIPID COMPOSITIONS FOR IMPROVING SLEEP

(71) Applicant: ENZYMOTEC LTD., Kfar Baruch (IL)

(72) Inventors: Fabiana BAR-YOSEPH, Haifa (IL); Yael LIFSHITZ, Zichron Yaakov (IL)

(73) Assignee: ENZYMOTEC LTD., Kfar Baruch (IL)

(21) Appl. No.: 15/571,661

(22) PCT Filed: May 4, 2016
(86) PCT No.: PCT/IL2016/050463

§ 371 Date: Nov. 3, 2017

Related U.S. Application Data
(60) Provisional application No. 62/156,378, filed on May 4, 2015.

(51) Int. Cl. A23L 33/115; A23L 33/00; A23D 9/04; A61K 31/202; A61K 31/201; C11C 3/08; C12P 7/64

(52) U.S. Cl. CPC A23L 33/115; A23L 33/40; A23D 9/04; A61K 31/202; A61K 31/231; C11C 3/08; C12P 7/6454; C12P 7/6472; A61K 31/201

(57) ABSTRACT

The present invention refers to a method for ore or both of improving sleep in a subject and treating at least one sleep problem in a subject. The method comprises administering to the subject an edible lipid composition which comprises a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid.

### FIELD OF THE INVENTION

The invention relates to lipid compositions and uses thereof in improving sleep.

### BACKGROUND OF THE INVENTION

Sleep patterns begin in utero, but are not fully developed until 4 to 6 months postbirth [1]. Newborns have a disorganized circadian rhythm for the first few weeks of life, with approximately equal amounts of sleep during the day and night. At age of about 2 months, night sleep begins to dominate over day sleep [2]. Melatonin and cortisol, circadian-driven hormones affecting sleep, are not endogenously produced until 3 months of age.

Infant sleep problems are a major parenteral concern which plays a significant role for complaints to clinicians during the first years of the child's life [3]. Research has shown that sleep problems in infants and young children are prevalent. Cross-sectional studies show that 15%-35% of all children aged between 6 months and 5 years are reported by their parents to show some type of sleep disturbance.

Some studies find no difference in sleep between breast fed and bottlefed infants while other studies suggest breastfeeding or bottle feeding might increase total sleep time or impact sleep.

The effects of the composition of human milk, formula, or additives on sleep quality and duration has been tested.

Steinberg et al. [4] and Yogman et al [5] demonstrate that feeding infants with formulas differing in tryptophan concentration produce differences in sleep latency, which could influence neurobehavioral development.

A randomised longitudinal study of 106 infants at ages 5 weeks and 4 months in which cereals were added to bottles of human milk or formula found no correlation between the cereal additive or feeding method and sleep [6].

WO 2009131939 [7] describes a lipid-containing composition which comprises omega6 and omega3 fatty acids in specific ratio for the preparation of dietary supplements for prophylaxis or treatment of disorders such as a sleep disorder.

CN 102524422 [8] describes an infant formula milk powder that has an effect of regulating sleep. The formula comprises vegetable oil or plant fat powder, fresh milk or dried skim milk, whey powder, lactose or glucose solid syrup, whey protein powder, oligosaccharide, a mineral premix, a vitamin premix, nutrient, and specific amount of alpha-lactalbumin and lactoferrin.

WO 2005/036987 [9] describes enzymatically prepared fat base compositions which comprise a mixture of vegetable-derived triglycerides, their preparation and various uses thereof.

Sleep plays a major role in children's well-being and is strongly influenced by the child's health status, psychological stress and family issues as well as by multiple aspects of his or her culture and environment. Also, children's sleep patterns affect their well-being within the same wide range of health and psychosocial phenomena. Sleep is also important for adults. Thus, improving sleep is of valuable importance.

### REFERENCES

[1] Bertelle, V., A. Sevestre, K. Laou-Hap, M. C. Nagahapitiye and J. Sizun. “Sleep in the neonatal intensive care unit” J Perinat Neonatal Nurs 21(2): 140-148; quiz 149-150, 2007.
[2] Pollak, C. P. “Regulation of sleep rate and circadian consolidation of sleep and wakefulness in an infant” Sleep 17(7): 567-575, 1994.
[3] Sadeh, A. and T. F. Andres. “Infant Sleep Problems: Origins, Assessment, Interventions” Infant Mental Health Journal 14(1), 1993.
[4] Steinberg, L. A., N. C. O'Connell, T. F. Hatch, M. F. Picciano and L. L. Birch. “Tryptophan intake influences infants' sleep latency” J Nutr 122(9): 1781-1791, 1992.
[5] Yogman, M. W. and S. H. Zeisel. “Diet and sleep patterns in newborn infants” N Engl J Med 309(19): 1147-1149, 1983.
[6] Macknin, M. L., S. V. Medendorp and M. C. Maier. “Infant sleep and bedtime cereal” Am J Dis Child 143(9): 1066-1068, 1989.
[7] WO 2009131939.
[8] CN 102524422.
[9] WO 2005/036987.

### SUMMARY OF THE INVENTION

The inventors of the present disclosure have surprisingly found that specific lipid compositions improve sleep patterns in infants administered with same. The lipid compositions may also be useful in treating sleep disorders in subjects such as infants.

Thus, the present invention provides, in accordance with the first of its aspects, an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in one or both of improving sleep in a subject and treating at least one sleep problem in a subject.

In a further one of its aspects, the present invention provides an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in a method for one or both of improving sleep in a subject and treating at least one sleep problem in a subject.

In yet a further aspect, the present invention provides an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for one or both of improving sleep in a subject and treating at least one sleep problem in a subject.

Yet, in a further aspect, the present invention provides an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or a nutraceutical composition or a functional or medical food, for one or more of improving sleep in a subject and treating at least one sleep problem in a subject.

In another one of its aspects the present invention provides a method comprising administering to a subject an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, the method being for one or both of improving sleep in a subject and treating at least one sleep problem in a subject.

In accordance with a further one of its aspects the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in one or both of improving sleep in a subject and treating at least one sleep problem in a subject.

In a further one of its aspects, the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in a method for one or both of improving sleep in a subject and treating at least one sleep problem in a subject.

In yet a further aspect, the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for one or both of improving sleep in a subject and treating at least one sleep problem in a subject.

Yet, in a further aspect, the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or a nutraceutical composition or a functional or medical food, for one or both of improving sleep in a subject and treating at least one sleep problem in a subject.

In another one of its aspects the present invention provides a method comprising administering to a subject an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, the method being for one or both of improving sleep in a subject and treating at least one sleep problem in a subject.

In another one of its aspects the present invention provides the lipid composition and/or the vegetable-derived fat source according to the invention for use in the preparation of a food article.

In another one of its aspects the present invention provides a food article, wherein the food article comprises the lipid composition and/or the vegetable-derived fat source in accordance with the invention, as described herein above and below, for (or for use in or for use in a method for) one or both of improving sleep in a subject and treating at least one sleep disorder in a subject.

In yet another one of its aspects the present invention provides a food article, wherein the food article comprises the lipid composition and/or the vegetable-derived fat source in accordance with the invention, as described herein above and below, for (or for use in or for use in a method for) improving sleep in a subject.

Yet in a further one of its aspects the present invention provides a food article, wherein the food article comprises the lipid composition and/or the vegetable-derived fat source in accordance with the invention, as described herein above and below, for (or for use in or for use in a method for) treating at least one sleep disorders in a subject.

In another one of its aspects the present invention provides a food article, wherein the food article comprises the lipid composition and/or the vegetable-derived fat source in accordance with the invention, as described herein above and below, for (or for use in or for use in a method for) improving sleep in a subject, particularly an infant, more particularly an infant at very early infancy e.g., newborn, infant at age of up to 6 weeks or up to 12 weeks or at the age of between about 6 to 12 weeks.

In another one of its aspects the present invention provides a food article, wherein the food article comprises the lipid composition and/or the vegetable-derived fat source in accordance with the invention, as described herein above and below, for (or for use in or for use in a method for) treating at least one sleep problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above.

In a further one of its aspects the present invention provides a formula (e.g., an infant formula, a baby formula, a toddler formula, a follow on formula, a child formula and an adult formula) comprising the lipid composition and/or the vegetable-derived fat source according to the invention for use in a method for one or both of improving sleep in a subject (e.g., an infant, a toddler, a child, an adult) and treating at least one sleep problem in a subject (e.g., an infant, a toddler, a child, an adult) administered with said formula.

In yet a further one of its aspects the present invention provides a formula (e.g., an infant formula, a baby formula, a toddler formula, a follow on formula, a child formula and an adult formula) comprising the lipid composition and/or the vegetable-derived fat source according to the invention for use in a method for improving sleep in a subject (e.g., an infant, a toddler, a child, an adult) administered with the formula.

Yet in a further one of its aspects the present invention provides a formula (e.g., an infant formula, a baby formula, a toddler formula, a follow on formula, a child formula and an adult formula) comprising the lipid composition and/or the vegetable-derived fat source according to the invention for use in a method for treating at least one sleep problem in a subject (e.g., an infant, a toddler, a child, an adult) administered with said formula.

In a further one of its aspects the present invention provides an infant formula comprising the lipid composition and/or the vegetable-derived fat source according to the invention for use in a method for one or both of improving sleep and treating at least one sleep problem said infant.

Yet, in a further one of its aspects the present invention provides an infant formula comprising the lipid composition and/or the vegetable-derived fat source according to the invention for use in a method for improving sleep in said infant.

Yet in a further one of its aspects the present invention provides an infant formula comprising the lipid composition and/or the vegetable-derived fat source according to the invention for use in a method for treating at least one sleep problem in infant.

In yet a further aspect, the present invention provides a commercial package comprising:

a) an edible lipid composition according to the invention and/or an edible vegetable-derived fat source according to the invention as herein disclosed which upon enteral administration to a subject improves sleep in a subject (e.g., an infant) and/or treats at least one sleep problem in a subject (e.g., an infant);
b) optionally, at least one of edible physiologically acceptable protein, carbohydrate, vitamin, mineral and active or non-active additives;
c) optionally, at least one edible physiologically acceptable carrier or diluent for carrying the constituent/s defined in a) and b);
d) means and receptacles for ad mixing the constituents defined in a), b) and/or c); and
e) instructions for use.

In another one of its aspects the present invention provides the compositions, fat sources, formulas, methods and uses as herein described and exemplified.

### DETAILED DESCRIPTION OF THE INVENTION

The invention provides a lipid composition for improving the sleep of subjects, specifically infants and children, in particular subjects whose sleep (e.g., sleep duration) is relatively shorter compared to the accepted norms for their age in healthy subjects.

The inventors of the present disclosure have shown that infants who were fed with the lipid composition according to the invention e.g., infant formula comprising suitable lipid composition as described herein, demonstrated increased sleep duration which was even more significant among infants who were defined as having sleep problems.

Thus, the present invention provides in accordance with its first aspect an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in one or both of improving sleep in a subject and treating at least one sleep problem in a subject.

Unless otherwise indicated, the percentages given throughout the text are provided in w/w. The term “w/w” refers to a weight per weight ratio.

The term “improving sleep” or any lingual variations thereof as used herein should be understood to encompass improvement of at least one sleep parameter. Non-limiting examples of such improvements are: improving sleep patterns, improving sleep maturation, improving rapid eye movement (REM) sleep, improving non-REM sleep, improving sleep quality, improving night sleep quality, improving sleep latency, increasing sleep duration, increasing night sleep duration, regulation of sleep, normalization of sleep e.g., normalization of sleep hours (as recommended for each age group of subjects e.g., infants) normalization of wake, reduce number of awakenings during sleep, improving continuous sleep, improving restful sleep, reducing fatigue symptoms, improving effectiveness of sleep and reducing night awaking, regulation of circadian rhythm. At times, the term is to be envisages as optimizing at least one sleep related parameter.

The term “sleep problem” or any lingual variations thereof as used herein should be understood to encompass a sleep disturbance, abnormal sleep related condition or a sleep disorder. Non-limiting examples of sleep problems include fatigue, insufficient REM sleep, REM Sleep behavior disorder, Stimulant-Dependent Sleep Disorder, Alcohol-Dependent Sleep Disorder, reduced sleeping hours (in comparison with accepted norms e.g., for a specific group of subjects such as infants at different ages), insufficient sleep duration, dyssomnias, parasomnias, sleep-onset difficulties, limit-setting sleep disorder, insufficient sleep syndrome, snoring and obstructive sleep apnea (OSA), sleepwalking, sleeplessness, insomnia, night terrors, nightmares, bedwetting, rhythmic movement disorders (such as head banging or rocking), restless leg syndrome, circadian rhythm disorders such as jet lag, shift work sleep disorder, delayed sleep phase, advanced sleep phase, non 24-hour sleep wake disorder, nocturnal sleep disturbance, awakening several times during the night, excessive tiredness related symptoms, problems falling asleep, problems remaining asleep, problem of experiencing restful sleep and irregular sleep-wake rhythm.

In the context of the present invention the term “treatment” or “treating” and the like are used herein to refer to obtaining a desired pharmacological or physiological effect on the subject, including prophylactic in terms of “preventing” or partially preventing an undesired condition or symptoms from developing and/or therapeutic in terms of “curing” partial or complete curing of an already existing undesired condition. The term “treating” is used within the context of the present disclosure as treatment of subjects who are healthy and/or suffer from a disorder, disease, or impaired physiological/medical sleep related condition. At times treating may be of a subject which may be one at risk for developing an undesired condition that may affect sleep.

In a further aspect, the present invention provides an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in a method for one or both of improving sleep in a subject and treating at least one sleep problem in a subject.

In yet a further aspect, the present invention provides an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for one or both of improving sleep in a subject and treating at least one sleep problem in a subject.

In yet another of its aspects, the present invention provides an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or nutraceutical composition, or a functional or medical food, for one or more of improving sleep in a subject and treating at least one sleep problem in a subject.

In a further aspect the invention provides a method comprising administering to a subject an edible lipid composition comprising a vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, the method being for one or both of improving sleep in a subject and treating at least one sleep problem in a subject.

In accordance with a further one of its aspects the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in one or both of improving sleep in a subject and treating at least one sleep problem in a subject.

In a further one of its aspects, the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in a method for one or both of improving sleep in a subject and treating at least one sleep problem in a subject.

In yet a further aspect, the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for one or both of improving sleep in a subject and treating at least one sleep problem in a subject.

Yet, in a further aspect, the present invention provides an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or a nutraceutical composition or a functional or medical food, for one or both of improving sleep in a subject and treating at least one sleep problem in a subject.

In another one of its aspects the present invention provides a method comprising administering to a subject an edible vegetable-derived fat source, wherein the fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, the method being for one or both of improving sleep in a subject and treating at least one sleep problem in a subject.

In another one of its aspects the present invention provides a food article, wherein the food article comprises the lipid composition and/or vegetable derived fat source in accordance with the invention, as described herein above and below, for (or for use in or for use in a method for) one or both of improving sleep in a subject and treating at least one sleep disorder in a subject.

In yet another one of its aspects the present invention provides a food article, wherein the food article comprises the lipid composition and/or the vegetable-derived fat source in accordance with the invention, as described herein above and below, for (or for use in or for use in a method for) improving sleep in a subject.

Yet in a further one of its aspects the present invention provides a food article, wherein the food article comprises the lipid composition and/or the vegetable-derived fat source in accordance with the invention, as described herein above and below, for (or for use in or for use in a method for) treating at least one sleep disorders in a subject.

In some embodiments according to the invention the food article may be selected from bakery products, including bread, particularly biscuits and pastries, human milk fat substitute, dairy products, including milk and dairy drinks, ice cream, cereal products, sauces, soup, spreads, including margarine, fillings, oils and fats, soy products, meat products, fried food products, confectionery products, bars, candy bars, candies and chocolates, snacks, drinks and shakes, instant products, instant drink products, frozen food, prepared foods for infants, toddlers and young children, including prepared cooked mashed vegetables and/or fruits, condiment products, and cooking oils and fats.

In the various aspects and embodiments of the invention the subject may suffer from a sleep problem. In some embodiments the sleep problem may be insufficient sleep duration, for example insufficient night sleep duration. In some embodiments the sleep problem may be reduced sleeping hours. Insufficiency of sleeping or reduced sleeping hours may be determined by comparison to acceptable values according to the norms.

As used herein, the term “subject” refers to a healthy subject or a subject suffering from a specific disorder/condition or at risk of developing a specific disorder/condition. It is noted that the disorder and/or condition may be related or may be unrelated to a sleep problem. Thus, at times, the subject may have a sleep problem which may be a side effect associated with a specific disorder and/or condition.

In some embodiments according to the invention the subject may be a subject in risk of developing a sleep problem. A subject in risk of developing a sleep problem may be, but is not limited to any one of: a preterm infant (born prematurely); a small for gestation age infant; a subject with poor sleeping patterns; children born prematurely; infants born by Caesarean section; infants suffering from colic; infants with feeding intolerance and abdominal pain or irritability; infants that need medical attention; infants with airway abnormalities; infants with breathing difficulties caused by gastroesophageal reflux or enlarged adenoids; a subject suffering from an endocrine malfunction (hormones); a subject suffering from a chronic illness; a subject with intrauterine sleep retardation or Intrauterine Growth Retardation (IUGR); a subject suffering from failure to thrive and/or inadequate weight gain anytime after birth; a subject consuming drugs that may affect sleep habits; a subject with Attention Deficit Hyperactivity Disorder (ADHD), a subject with breathing disorders; a subject with dementia; a subject suffering from depression; a subject with Chronic obstructive pulmonary disease (COPD); a subject suffering from epilepsy; a subject suffering from pain, etc.

In the various aspects and embodiments of the invention the subject may by a human infant, a human toddler, a human child, an adolescent, an adult and a geriatric subject.

In some embodiments according to the invention the subject may or may not suffer from medical problems that may interfere with sleep.

In some embodiments according to the invention the subject may be any one of an infant (preterm or term, newborn from the day of birth, to age of about 12 months i.e., about 1 year), a toddler (from about one year up to about the age of 3), a child (from about 3 years to about 12 years), an adolescent (from 12 years to about 18 years), an adult (over 18 years). At times the subject may be a geriatric subject.

In some embodiments according to the invention the infant may be any one of pre-term infant and term infant; a small for gestation age (SGA) infant; an appropriate for gestation age (AGA) infant; large for gestation age (LGA) infant; an infant born by regular (vaginal) delivery, cesarean surgery (Caesarean section) or any other modes of delivery.

As used herein, the term “newborn” includes pre-mature infants, post-mature infants and full term newborns.

In some embodiments of the invention the infant may be an infant at very early infancy e.g., newborn, infant at age of up to 6 weeks or up to 12 weeks or at the age of between about 6 to 12 weeks. In some embodiments of the invention the infant may be at the age of 12 weeks or above, at times at the age of between about 12 to 24 weeks and even at times at the age of 24 weeks or above. Further, at times the infant may be at the age of between about 24 weeks to about 12 months.

In the various aspects and embodiments of the invention the subject may be a human adult.

In some embodiments according to the invention the subject is an infant, a child or an adult. In some further embodiments according to the invention the subject is a child or an infant.

In some embodiments of the invention the subject is an infant.

In some embodiments of the invention the subject is a preterm infant.

In some embodiments of the invention the subject is a toddler.

In some embodiments of the invention the subject is a child.

In some embodiments of the invention the subject is an adolescent.

In some embodiments of the invention the subject is an adult.

In some embodiments of the invention the subject is a geriatric subject.

In some embodiments of the invention the subject may be a healthy subject.

In some embodiments of the invention the subject may be a non-healthy subject.

In the various aspects and embodiments of the invention, the lipid composition and/or the vegetable-derived fat source according to the invention may be provided to the infant for a period of time from day one to weeks, months, years, etc. following birth.

In some embodiments of the invention the subject may be a healthy subject experiencing sleep problem/s such as for example reduced sleeping hours (e.g., insufficient sleep such as insufficient night sleep duration). The subject may also be a non-healthy subject experiencing sleep problem/s such as for example reduced sleeping hours (e.g., insufficient sleep like insufficient night sleep duration).

In some embodiments according to the invention the subject may experience one or more sleep problem/s.

In a specific embodiment of the invention the lipid composition and/or the vegetable-derived fat source of the invention is for (or for use, or for use in a method for) improving sleep in a subject, particularly an infant, more particularly an infant at very early infancy e.g., newborn, infant at age of up to 6 weeks or up to 12 weeks or at the age of between about 6 to 12 weeks.

In a specific embodiment the methods and/or uses according to the invention are for improving sleep in a subject, particularly an infant, more particularly an infant at very early infancy e.g., newborn, infant at age of up to 6 weeks or up to 12 weeks or at the age of between about 6 to 12 weeks.

In another one of its aspects the present invention provides a food article, wherein the food article comprises the lipid composition and/or the vegetable-derived fat source in accordance with the invention, as described herein above and below, for (or for use in or for use in a method for) improving sleep in a subject, particularly an infant, more particularly an infant at very early infancy e.g., newborn, infant at age of up to 6 weeks or up to 12 weeks or at the age of between about 6 to 12 weeks.

In some embodiments, the compositions, fat sources, methods or uses disclosed herein are for improving sleep in an infant, wherein improving sleep being increasing sleep duration e.g., night sleep duration, or increasing sleeping hours (during the whole day or during the night).

In a specific embodiment of the invention the edible lipid composition of the invention and/or the vegetable-derived fat source of the invention is for (or for use, or for use in a method for) treating at least one sleep problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above.

In a specific embodiment the method of the invention is for treating at least one sleep problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above.

In another one of its aspects the present invention provides a food article, wherein the food article comprises the lipid composition and/or the vegetable-derived fat source in accordance with the invention, as described herein above and below, for (or for use in or for use in a method for) treating at least one sleep problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above.

In some embodiments, the compositions, fat sources, methods or uses disclosed herein are for treating at least one sleep problem in an infant, wherein the sleep problem is insufficient sleeping duration e.g., insufficient night sleep duration and/or reduced sleeping hours e.g., reduced night sleeping hours compared for example to acceptable norms at the specific subject's gender and/or age and/or weight and/or health condition and the like.

In some embodiments according to the invention the subject may be a healthy subject (e.g., an infant, a toddler, a child, an adolescent and/or an adult) suffering from insufficient sleep (e.g., insufficient night sleep duration) compared for example to acceptable norms at the specific subject's gender and/or age and/or weight and/or health condition.

In some embodiments according to the invention the subject may be a non-healthy subject (e.g., an infant, a toddler, a child, an adolescent and/or an adult) with insufficient sleep (e.g., insufficient night sleep duration) compared for example to acceptable norms at the specific subject's gender and/or age and/or weight and/or health condition.

In some embodiments of the invention the subject may suffer from reduced sleeping hours. To this end, the problem may be reduced sleeping hours or insufficient sleep duration.

As used herein, the term “increase sleep duration” or any lingual variations thereof is to be envisaged as any one of, but not limited to, increasing the time duration of a single sleeping episode, increasing accumulated sleeping time during the whole day (out of 24 hours) and increasing accumulated sleeping time during the night.

As used herein, the term “insufficient sleep duration” or any lingual variations thereof is to be envisaged as any one of, but not limited to, insufficient time duration of a single sleeping episode, insufficient accumulated sleeping time during the whole day (out of 24 hours) and insufficient accumulated sleeping time during the night compared for example to acceptable norms at the specific subject's gender and/or age and/or weight and/or health condition.

In some embodiments of the invention the compositions, fat sources, formulas, food articles and methods according to the invention are for improving sleep.

In some embodiments of the invention the compositions, fat sources, formulas, food articles and methods according to the invention are for treating at least one sleep problem.

In some embodiments of the invention the compositions, fat sources, formulas, food articles and methods according to the invention are for increasing sleep duration.

In some embodiments of the invention the compositions, fat sources, formulas, food articles and methods according to the invention are for increasing night sleep duration. In the various aspects and embodiments of the invention, the triglycerides according to the invention may comprise saturated and/or mono-unsaturated and/or poly-unsaturated fatty acids residues.

In the various aspects and embodiments of the invention, the fatty acid residues at the sn-2 position of the glycerol backbone may be a saturated fatty acid residue, including C₈ to C₂₄, and in some particular embodiments C₁₄-C₁₈ fatty acid residues.

In the various aspects and embodiments of the invention, the saturated fatty acid may be any one of butyric acid (butanoic acid, C4:0), caproic acid (hexanoic acid, C6:0), caprylic acid (octanoic acid, C8:0), capric acid (decanoic acid, C10:0), lauric acid (dodecanoic acid, C12:0), myristic acid (tetradecanoic acid, C14:0), palmitic acid (hexadecanoic acid, C16:0), stearic acid (octadecanoic acid, C18:0), arachidic acid (eicosanoic acid, C20:0) and behenic acid (docosanoic acid C22:0).

In the various aspects and embodiments of the invention an unsaturated fatty acid may be any one of oleic acid (C18:1), linoleic acid (C18:2), α-linolenic acid (C18:3) and gadoleic acid (C20:1).

In some specific embodiments, according to all aspects of the invention, the saturated fatty acid residue is predominantly a palmitic acid residue.

In the various aspects and embodiments of the invention, in the vegetable-derived fat source according to the invention at least about 30%°, at time, at least about 33%, at times at least about 38%, at times at least about 40%, at times at least about 43%, at times at least about 44% and even at times at least about 50% of the total palmitic acid residues are present at the sn-2 position of the glycerol backbone. In some further embodiments of the invention the level of palmitic acid moieties at the sn-2 position of the glycerol backbone is 30%-70% of total palmitic acid. In yet some further embodiments of the invention the level of palmitic acid moieties at the sn-2 position of the glycerol backbone is 40%-70% of total palmitic acid. In yet some further embodiments of the invention the level of palmitic acid moieties at the sn-2 position of the glycerol backbone is 40%-60% of total palmitic acid. In some further embodiments of the invention the level of palmitic acid moieties at the sn-2 position of the glycerol backbone is 43%-60%, at times 44%-55% of total palmitic acid. In yet some further embodiments of the invention the level of palmitic acid moieties at the sn-2 position of the glycerol backbone is 50%-65%, at times 55%-70% of total palmitic acid. In yet some further embodiments of the invention the level of palmitic acid moieties at the sn-2 position of the glycerol backbone is 40%-55%, at times 40%-50% of total palmitic acid.

In the various aspects and embodiments of the invention, in the vegetable-derived fat source according to the invention at least about 20%, at times at least about 23%, at times at least about 30%, at times at least about 40%, at times at least about 44%, at times 40%-80%, at times 44%-70%, at times 50%-55% of the total fatty acid moieties at the sn-2 position of the glycerol backbone are saturated.

In some embodiments according to the invention, in the vegetable-derived fat source according to the invention at least about 20% of the total fatty acid moieties at the sn-2 position of the glycerol backbone are saturated.

In the various aspects and embodiments of the invention, in the vegetable-derived fat source according to the invention at least about 10%, at times at least about 15%, at times at least about 20%, at times at least about 22%, at times at least 27%, at times 15%-65%, at times 20%-40%, at times 20%-35%, at times 20%-30%, at times 27%-40% of the total fatty acid moieties at the sn-2 position of the glycerol backbone are palmitic acid.

In the various aspects and embodiments of the invention, in the vegetable-derived fat source according to the invention at most about 40%, at times at most about 36%, at times at most about 20%, at times at most 17%, at times 5%-40%, at times 9%-36% of the total fatty acid moieties at the sn-1 and sn-3 positions of the glycerol backbone are palmitic acid.

In the various aspects and embodiments of the invention, in the vegetable-derived fat source according to the invention at most about 70%, at times at most about 60%, at times at most about 55%, at times at most about 47%, at times 30%-70%, at times 40%-65%, at times 41%-65%, at times 43%-61%, of the total palmitic acid are esterified at the sn-1 and sn-3 positions of the glycerol backbone.

In the various aspects and embodiments of the invention, in the vegetable-derived fat source according to the invention at most about 80%, at times at most about 60%, at times at most 50%, at times at most about 45%, at times at most about 40%, at times at most about 38%, at times at most about 33%, at times at most about 25%, at times at most about 15%, at times 10-50%, at times 10-45%, at times 14-40%, at times 25%-50%, at times 40%-50% of the total fatty acid moieties, at the sn-1 and sn-3 positions of the glycerol backbone are saturated.

In the various aspects and embodiments of the invention, in the vegetable-derived fat source according to the invention at least about 45%, at least about 50%, at times at least about 60%, at times at least about 70%, at times 50-80%, at times 60%-80%, at times 60-75%, at times 65%-70% of the total fatty acid moieties at the sn-1 and sn-3 positions of the glycerol backbone are unsaturated.

In the various aspects and embodiments of the invention, in the vegetable-derived fat source according to the invention at most about 80%, at times at most about 70%, at times at most about 60%, at times 25%-70%, at times 28%-40% of the total fatty acid moieties at the sn-2 position of the glycerol backbone are unsaturated.

In the various aspects and embodiments of the invention, in the vegetable-derived fat source according to the invention at least about 40%, at times at least about 50%, at times at least about 60%, at times 40-80%, at times 50%-65% of the fatty acids are unsaturated fatty acid moieties.

In some embodiments according to the present invention, in the vegetable-derived fat source according to the invention at least about 40% of the fatty acids are unsaturated fatty acid moieties.

In the various aspects and embodiments of the invention, in the vegetable-derived fat source according to the invention at most about 1%, at times at most about 0.5%, at times at most about 0.2%, at times at most 0.1% of the total fatty acid moieties are free fatty acids.

In the various aspects and embodiments of the invention, in the vegetable-derived fat source according to the invention at most about 5%, at times at most about 4%, at times at most about 3%, at times at most 2% of the total fatty acid moieties are in form of diacylglycerides.

In the various aspects and embodiments of the invention, in the vegetable-derived fat source according to the invention at most about 3%, at times at most about 1%, at times at most about 0.5%, at times at most 0.1% of the total fatty acid moieties are in form of monoacylglycerides.

In the various aspects and embodiments of the invention, in the vegetable-derived fat source according to the invention at least about 50%, at times, at least 65% of the unsaturated fatty acids are oleic acid, at times 50%-90%, at times 70%-90% of the unsaturated fatty acids are oleic acid.

In some embodiments according to the invention, in the vegetable-derived fat source according to the invention at least about 50% of the unsaturated fatty acids are oleic acid.

In some embodiments of the invention, in the vegetable-derived fat source according to the invention at least about 5%, at times at least 10%, at times at least about 13%, at times at least about 15%, at times at least 20%, at times at least about 46%, at times 10%-60%, at times 10%-50%, at times 15%-55%, at times 15%-35%, at times 10%-26%, at times 10%-15%, at times 40%-50% of the unsaturated fatty acids are linoleic acid.

In some embodiments according to the present invention, in the vegetable-derived fat source according to the invention at least about 10% of the unsaturated fatty acids are linoleic acid.

In some further embodiments of the invention, in the vegetable-derived fat source according to the invention at least about 1%, at times, at least 2.5%, at times 1%-8%, at times 0%-3.5%, at times 0%-1% of the unsaturated fatty acids are linolenic acid.

In some embodiments of the invention, in the vegetable-derived fat source according to the invention at least about 1% of the unsaturated fatty acids are linolenic acid.

In some further embodiments of the invention, in the vegetable-derived fat source according to the invention at most about 5%, at times at most about 3.5%, at times at most 2.5%, at times at most 2%, at times 0.1%-8%, at times 0.1%-4%, at times 0.1%-3.5%, at times 0.2%-2.5% and at times 0.4%-2% of the unsaturated fatty acids are linolenic acid.

In some embodiments according to the present invention, in the vegetable-derived fat source according to the invention at most about 5% of the unsaturated fatty acids are linolenic acid.

In the various aspects and embodiments of the invention, in the vegetable-derived fat source according to the invention at most about 35%, at times 10%-35%, at times 20%-35% of the total fatty acid moieties at the sn-2 position of the glycerol backbone are oleic acid.

In the various aspects and embodiments of the invention, in the vegetable-derived fat source according to the invention at most about 35%, at times at most about 27%, at times 15%-35%, at times 20%-30% of the total oleic acid moieties are at the sn-2 position of the glycerol backbone.

In the various aspects and embodiments of the invention, in the vegetable-derived fat source according to the invention at most about 80%, at times at most about 76%, at times at most about 65%, at times 20%-76%, at times 25%-76%, at times 25%-55%, at times 35%-55% of the total fatty acid moieties at the sn-2 position of the glycerol backbone are unsaturated fatty acids.

In some embodiments according to the present invention, in the vegetable-derived fat source according to the invention at least about 45% of the total fatty acid moieties at the sn-1 and sn-3 positions of the glycerol backbone are unsaturated.

In the various aspects and embodiments of the invention, in the vegetable-derived fat source according to the invention at least about 35%, at times at least about 40%, at times at least about 57%, at times at least about 70%, at times at least 75% of the unsaturated fatty acid moieties at the sn-1 and sn-3 positions are oleic acid moieties. In some further embodiments of the invention, in the vegetable-derived fat source according to the invention 40%-90%, at times 50%-90%, at times 60%-80%, at times 70%-90% of the unsaturated fatty acid moieties at the sn-1 and sn-3 positions are oleic acid moieties.

In some embodiments according to the present invention, in the vegetable-derived fat source according to the invention at least about 35% of the unsaturated fatty acid moieties at the sn-1 and sn-3 positions are oleic acid moieties.

In the various aspects and embodiments of the invention, in the vegetable-derived fat source according to the invention at least about 4%, at times at least about 6%, at times at least about 10%, at times at least about 12%, at times at least about 22%, at times 6%-50%, at times 6%-30%, at times 10%-45%, at times 10%-40%, at times 10%-30%, at times 10%-35%, at times 10%-15% of the unsaturated fatty acid moieties at the sn-1 and sn-3 positions are linoleic acid moieties.

In some embodiments according to the present invention, in the vegetable-derived fat source according to the invention at least about 4% of the unsaturated fatty acid moieties at the sn-1 and sn-3 positions are linoleic acid moieties.

In the various aspects and embodiments of the invention, the vegetable-derived fat source according to the invention is characterized by having the following parameters: (i) at least 20%, at times at least 30%, at times, at least 33%, at times at least 38%, and even at times at least 40% of the total palmitic acid residues are at the sn-2 position of the glycerol backbone; (ii) at least 50%, at times at least 60%, at times, at least 70% of the fatty acid moieties at the sn-1 and sn-3 positions of the glycerol backbone are unsaturated; (iii) at least 35%, at times at least 40%, at times at least about 50%, at times at least about 60%, at times at least about 70% of the unsaturated fatty acid moieties at the sn-1 and sn-3 positions are oleic acid moieties; and (iv) at least 4%, at times at least 6%, at times at least 10%, at times at least 22% of the unsaturated fatty acid moieties at the sn-1 and sn-3 positions are linoleic acid moieties.

In the various aspects and embodiments of the invention, the vegetable-derived fat source comprises triglycerides with at most 50%, at times at most 38%, at times about 10%-50%, at times about 15%-50%, at times 15%-40%, at times 15%-38%, at times 15%-33%, at times 15%-25%, at times 17%-24%, at times 17%-23%, at times 18%-23%, at times 18%-22%, at times 19%-22% and at times 19%-21%, palmitic acid moieties out of the total fatty acids. In some embodiments of the invention the vegetable-derived fat source comprises triglycerides with about 20%-50%, at times 25%-40%, at times 30%-40%, at times 30%-38%, at times 30%-35%, at times 31%-34% and at times 31%-33% palmitic acid moieties out of the total fatty acids.

Thus, the palmitic acid content of the fat source according to the invention may be 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54% or 55% of the total fatty acids.

In the various aspects and embodiments of the invention, in the vegetable-derived fat source according to the invention at least about 13% w/w, at times at least about 15%, at times, at least about 18%, at times at least about 22%, at times 18%-60%, at times 22%-50%, at times 20%-45%, at times 40%-50% of the total fatty acid residues at the sn-2 position of the glycerol backbone are palmitic acid residues. In some embodiments of the invention, in the vegetable-derived fat source according to the invention at least 50%, at times at least 60%, at times 50%-80%, at times 60%-90%, at times 60%-70% of the fatty acid moieties at the sn-2 position of the glycerol backbone are palmitic acid residues.

In the various aspects and embodiments of the invention, in the vegetable-derived fat source the level of palmitic acid moieties at the sn-2 position of the glycerol backbone is 40%-60% of total palmitic acid, 20%-30% of the total fatty acid moieties at the sn-2 position of the glycerol backbone are palmitic acid, 45%-61% of the total palmitic acid are esterified at the sn-1 and sn-3 positions of the glycerol backbone, 23%-35% of the total fatty acid moieties at the sn-2 position of the glycerol backbone are oleic acid, 17%-30% of the total oleic acid moieties are at the sn-2 position of the glycerol backbone, 10%-45% of the total fatty acid moieties, at the sn-1 and sn-3 positions of the glycerol backbone are saturated and 45%-80% of the total fatty acid moieties at the sn-1 and sn-3 positions of the glycerol backbone are unsaturated.

A non-limiting example of a lipid composition or the vegetable-derived fat source according to all aspects of the invention comprises:

0%-10%—C8:0 fatty acid residue out of the total fatty acid residue content;
0%-10%—C10:0 fatty acid residue out of the total fatty acid residue content;
0%-22%—C12:0 fatty acid residue out of the total fatty acid residue content;
0%-15%—C14:0 fatty acid residue out of the total fatty acid residue content;
15%-55%—C16:0 fatty acid residue out of the total fatty acid residue content;
1%-13%—C18:0 fatty acid residue out of the total fatty acid residue content;
20%-75%—C18:1 fatty acid residue out of the total fatty acid residue content;
2%-40%—C18:2 fatty acid residue out of the total fatty acid residue content; and
0%-8%—C18:3 fatty acid residue out of the total fatty acid residue content, and
wherein at least 20%, at times at least 30%, at times at least 33%, and even at times at least 40%, at times 40%-70%, even at times 40%-60% or 43%-60% of the C16:0 fatty acid residue out of the total fatty acid residue content is at sn-2 position the glycerol backbone.

A further non-limiting example of a lipid composition or the vegetable-derived fat source according to all aspects of the invention comprises:

0%-10%—C8:0 fatty acid residue out of the total fatty acid residue content;
0%-10%—C10:0 fatty acid residue out of the total fatty acid residue content;
0%-22%—C12:0 fatty acid residue out of the total fatty acid residue content;
0%-15%—C14:0 fatty acid residue out of the total fatty acid residue content;
15%-55%—C16:0 fatty acid residue out of the total fatty acid residue content;
1%-7%—C18:0 fatty acid residue out of the total fatty acid residue content;
20%-75%—C18:1 fatty acid residue out of the total fatty acid residue content;
2%-40%—C18:2 fatty acid residue out of the total fatty acid residue content; and
0%-8%—C18:3 fatty acid residue out of the total fatty acid residue content, and
wherein at least 20%, at times at least 30%, at times at least 33%, and even at times at least 40%, at times 40%-70%, even at times 40%-60% or 43%-60% of the C16:0 fatty acid residue out of the total fatty acid residue content is at sn-2 position the glycerol backbone.

In accordance with a more particular embodiment, the lipid composition or the vegetable-derived fat source according to all aspects of the invention comprises:

0%-2%—C8:0 fatty acid residue out of the total fatty acid residue content;
0%-2%—C10:0 fatty acid residue out of the total fatty acid residue content;
5%-15%—C12:0 fatty acid residue out of the total fatty acid residue content;
2%-10%—C14:0 fatty acid residue out of the total fatty acid residue content;
17%-25%—C16:0 fatty acid residue out of the total fatty acid residue content;
2%-5%—C18:0 fatty acid residue out of the total fatty acid residue content;
28%-48%—C18:1 fatty acid residue out of the total fatty acid residue content;
5%-20%—C18:2 fatty acid residue out of the total fatty acid residue content;
1%-3%—C18:3 fatty acid residue out of the total fatty acid residue content; and
wherein at least 20%, at times at least 30%, at times at least 33%, and even at times at least 40%, at times 40%-70%, even at times 40%-60% or 43%-60% of the C16:0 fatty acid residue out of the total fatty acid residue content is at sn-2 position the glycerol backbone.

Thus, the vegetable-derived fat source according to the invention may comprise: 0%, 1%, 2%, 3%, 4%, 5%, 6%, 7%, 8%, 9% or 10% of C8:0 fatty acids of the total fatty acids; 0%, 1%, 2%, 3%, 4%, 5%, 6%, 7%, 8%, 9% or 10% of C12:0 fatty acids of the total fatty acids; 0%, 1%, 2%, 3%, 4%, 5%, 6%, 7%, 8%, 9%, 10%, 11%, 12%, 13%, 14%, 15%, 16%, 17%, 18%, 19%, 20%, 21% or 22% of C12:0 fatty acids of the total fatty acids; 0%, 1%, 2%, 3%, 4%, 5%, 6%, 7%, 8%, 9%, 10%, 11%, 12%, 13%, 14% or 15% C14:0 fatty acids of the total fatty acids; 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54% or 55% C16:0 fatty acids of the total fatty acids; 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, 5%, 5.2%, 5.4%, 5.6%, 5.8%, 6%, 6.2%, 6.4%, 6.6%, 6.8%, 7%, 8%, 9%, 10%, 11%, 12% or 13% C18:0 fatty acids of the total fatty acids; 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75% C18:1 fatty acids of the total fatty acids; 2%, 3%, 4%, 5%, 6%, 7%, 8%, 9%, 10%, 11%, 12%, 13%, 14%, 15%, 16%, 17%, 18%, 19%, 20%, 22%, 23%, 25%, 26%, 27%, 28%, 29%, 30%, 31%, 32%, 33%, 34%, 35%, 36%, 37%, 38%, 39%, 40% C18:2 fatty acids of the total fatty acids; 0%, 0.5%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, 5%, 5.2%, 5.4%, 5.6%, 5.8%, 6%, 6.2%, 6.4%, 6.6%, 6.8%, 7%, 7.2%, 7.5%, 7.8% or 8% C18:3 fatty acids of the total fatty acids.

Of particular interest are vegetable-derived fat sources which are based on edible synthetic oils (which can be enzymatically produced), which mimic, as are (designated herein as fat base), or when blended with edible vegetable oil/s (designated herein as fat blend) the triglyceride composition of human breast milk fat. Such fat sources have a high level of palmitic acid at the sn-2 position of the triglycerides, and a high level of unsaturated fatty acids at sn-1 and sn-3 positions.

The edible vegetable oil/s which may be blended with said fat base to prepare the fat blend may be: natural vegetable oil/s, randomized vegetable oil/s, interesterified vegetable oils, enzymatically interesterified vegetable oils, at least two vegetable oils which were co-randomized, at least two vegetable oils which were co-interesterified. Said edible vegetable oil/s comprises oils selected from the group consisting of but not limited to soybean oil, high oleic sunflower oil, high oleic safflower oil palm oil, palm olein oil, canola oil, coconut oil, palm kernel oil, sunflower oil, corn oil, safflower and rapeseed oil.

The lipid composition and/or vegetable-derived fat source of the invention may be used in the preparation of pharmaceutical, nutritional or nutraceutical composition, or a functional or medical food.

The lipid composition and/or vegetable-derived fat source of the invention may be used in the preparation of infant formulas, toddler formula, child formula, adolescent formula, dietary supplements and food articles.

In some embodiments the lipid composition and/or vegetable-derived fat source of the invention may be used in the preparation of infant formula.

In some embodiments the lipid composition and/or vegetable-derived fat source of the invention may be used in the preparation of toddler formula.

In some embodiments the lipid composition and/or vegetable-derived fat source of the invention may be used in the preparation of a follow on formula i.e., a formula for subjects at the ages of 6 months to 2 years.

In some embodiments the lipid composition and/or vegetable-derived fat source of the invention may be used in the preparation of child formula.

In some embodiments the lipid composition and/or vegetable-derived fat source of the invention may be used in the preparation of adolescent formula.

In some embodiments the lipid composition and/or vegetable-derived fat source of the invention may be used in the preparation of adult formula.

In some embodiments the lipid composition and/or vegetable-derived fat source of the invention may be used in the preparation of geriatric formula.

Thus, in the various aspects and embodiments of the invention, the vegetable-derived fat source may be man-made, synthetically made, artificially made and/or enzymatically made.

Further, in the various aspects and embodiments of the invention, the vegetable-derived fat source and/or at least one triglyceride of the fat source may be selected from the group consisting of naturally occurring triglycerides, synthetic triglycerides, semi-synthetic triglycerides, and artificially produced triglycerides. In some further embodiments the triglyceride may be obtained from a vegetable source.

The vegetable-derived fat source according to the invention can also be a substitute human milk fat composition or human milk fat mimetic composition comprising a blend of at least 25%, at times 25-83%, at times 30-70% of fat base concentrate and up to 75%, at times 17-75%, at times 20-75%, at times 30-70% of at least one edible vegetable oil. In some specific embodiments the fat source may comprise 25%, 30%, 36%, 50%, 52%, 60%, 63%, 73% and 83% of the fat base concentrate and 75%, 70%, 64%, 50%, 48%, 40%, 37%, 27% and 17%, respectively, of the at least one vegetable oil.

Most importantly, the lipid composition and/or vegetable-derived fat source of the present invention may be used in the preparation of infant formula, toddler formula or child formula.

Thus, in a further one of its aspects the present invention provides a formula comprising the lipid composition and/or vegetable-derived fat source according to the invention for use in a method for one or both of improving sleep in a subject) and treating at least one sleep problem in a subject administered with said formula.

In some embodiments according to the invention the formula is a preterm infant formula. In some embodiments according to the invention the formula is an infant formula. In some embodiments according to the invention the formula is a toddler formula. In some embodiments according to the invention the formula is a child formula. In some embodiments according to the invention the formula is an adolescent formula. In some embodiments according to the invention the formula is an adult formula. In some embodiments according to the invention the formula is a geriatric formula.

In a further one of its aspects the present invention provides a formula (e.g., an infant formula, a baby formula, a toddler formula, a follow on formula, a child formula and an adult formula) comprising the lipid composition and/or the vegetable-derived fat source according to the invention for use in a method for improving sleep in a subject (e.g., an infant, a toddler, a child, an adult) administered with the formula.

In yet a further one of its aspects the present invention provides a formula (e.g., an infant formula, a baby formula, a toddler formula, a follow on formula, a child formula and an adult formula) comprising the lipid composition and/or the vegetable-derived fat source according to the invention for use in a method for treating at least one sleep problem in a subject (e.g., an infant, a toddler, a child, an adult) administered with said formula.

In a further one of its aspects the present invention provides an infant formula comprising the lipid composition and/or the vegetable-derived fat source according to the invention for use in a method for one or both of improving sleep and treating at least one sleep problem said infant.

In yet a further one of its aspects the present invention provides an infant formula comprising the composition and/or vegetable-derived fat source according to the invention for use in a method for improving sleep in said infant administered with said formula.

Yet in a further one of its aspects the present invention provides an infant formula comprising the composition and/or vegetable-derived fat source according to the invention for use in a method for treating at least one sleep problem in said infant administered with said formula.

In yet a further one of its aspects the present invention provides a toddler formula (e.g., follow on formula) comprising the composition and/or vegetable-derived fat source according to the invention for use in a method for improving sleep in said toddler administered with said formula.

Yet in a further one of its aspects the present invention provides a toddler formula (e.g., follow on formula) comprising the composition and/or vegetable-derived fat source according to the invention for use in a method for treating at least one sleep problem in said toddler administered with said formula.

In yet a further one of its aspects the present invention provides a child formula (e.g., follow on formula) comprising the composition and/or vegetable-derived fat source according to the invention for use in a method for improving sleep in said child administered with said formula.

Yet in a further one of its aspects the present invention provides a child formula (e.g., follow on formula) comprising the composition and/or vegetable-derived fat source according to the invention for use in a method for treating at least one sleep problem in said child administered with said formula.

In yet a further one of its aspects the present invention provides an adult formula comprising the composition and/or vegetable-derived fat source according to the invention for use in a method for improving sleep in said adult administered with said formula.

Yet in a further one of its aspects the present invention provides an adult formula comprising the composition and/or vegetable-derived fat source according to the invention for use in a method for treating at least one sleep problem in said adult administered with said formula.

In yet a further one of its aspects the present invention provides a geriatric formula comprising the composition and/or vegetable-derived fat source according to the invention for use in a method for improving sleep in said geriatric subject administered with said formula.

Yet in a further one of its aspects the present invention provides a geriatric formula comprising the composition and/or vegetable-derived fat source according to the invention for use in a method for treating at least one sleep problem in said geriatric subject administered with said formula.

The formula used by the invention may comprise in addition to the fat source at least one protein component and optionally at least one of carbohydrate source, vitamins, minerals, nucleotides and amino acids.

Thus, in the various aspects and embodiments of the invention, the formula (e.g., infant formula) comprises the lipid composition and/or vegetable-derived fat source, together with a protein source, a carbohydrate source, minerals, vitamins and optionally at least one of carrier, diluent, additive or excipient.

The lipid composition and/or vegetable-derived fat source of the present invention may be used in the preparation of a food article. The food article used by the invention comprises in addition to the fat source at least one protein component and optionally at least one of carbohydrate source, vitamins, minerals, and amino acids.

Thus, in the various aspects and embodiments of the invention, the food article comprises the lipid composition and/or vegetable-derived fat source, together with a protein source, a carbohydrate source, minerals, vitamins and optionally at least one of carrier, diluent, additive or excipient.

The lipid composition and/or vegetable-derived fat source of the present invention may be used in the preparation of a formula (e.g., infant, child and adult). The formula used by the invention comprises in addition to the fat source at least one protein component and optionally at least one of carbohydrate source, vitamins, minerals, and amino acids.

Thus, in the various aspects and embodiments of the invention, the formula (e.g., infant, child and adult) comprises the lipid composition and/or vegetable-derived fat source, together with a protein source, a carbohydrate source, minerals, vitamins and optionally at least one of carrier, diluent, additive or excipient.

The terms “lipid” and “fat” are used herein synonymously.

The methods according to the invention are best practiced through administering to a subject, an infant formula, toddler formula, child formula, adult formula, clinical nutrition product or a food article prepared with and comprising the lipid composition and/or vegetable-derived fat source as described in the invention, either in the form of a concentrate base or in the form of a blend. Non-limiting examples of a fat concentrate/base are Fat Bases 1 to 11 in Table 1 of Example 1, and non-limiting examples of blends are described in Table 2-6 of Example 1 as well as in Examples 2 and 3.

The lipid composition and/or vegetable-derived fat source according to the invention may be used in the preparation of or as part of a nutritional, pharmaceutical, or nutraceutical composition or a functional or medical food.

A nutritional composition as used herein can be any nutritional composition including, but not limited to: human milk fat substitute, formula, infant formula, toddler formula, child formula, adult formula, dairy product including milk and dairy drinks, milk powder, drinks, shakes, ice cream, biscuit, soy product, bakery, pastry, bread, cake, sauce, soup, prepared food, including prepared mashed vegetables and/or fruits, frozen food, condiment, confectionary, oil, fat, margarine, spread, filling, meat product, cereal, instant product, instant drink product, infant food, toddler food, bar, snack, candy, and chocolate product.

A functional food as used herein can be any functional food, including, but not limited to: dairy product, ice-cream, biscuit, soy product, bakery, pastry, cakes and bread, instant product, sauce, soup, prepared food, frozen food, condiment, confectionary, oils and fat, margarine, spread, filling, cereal, instant product, drinks and shake, infant food, bar, snack, candy, and chocolate product.

A nutraceutical composition as used herein can be any nutraceutical, which can be any substance that may be considered as a food or part of a food and provides medical or health benefits, including the prevention and treatment of diseases or disorders. Such nutraceutical compositions include, but are not limited to: a food additive, a food supplement, a dietary supplement, genetically engineered foods (such as for example vegetables, herbal products, and processed foods such as cereals, soups, and beverages), stimulant functional food, clinical nutrition product, medical food, and pharmafood. Dietary supplements may be delivered in the form of soft gel capsules, tablets, syrups, and other known dietary supplement delivery systems.

The pharmaceutical or nutraceutical compositions may be in any of the many dosage delivery forms commonly used in the art. Pharmaceutical compositions suitable for oral administration may be presented as discrete dosage units (such as pills, tablets, pellets, dragees, capsules, or softgel), as a powder or granule, or as a solution, suspension, syrup, or elixir.

A medical food as used herein is specially formulated and intended for the dietary management of a disease/disorder that has distinctive nutritional needs that cannot be met by normal diet alone.

According to a specific embodiment of the invention, provided are infant formulas, toddler formulas, child formula and/or adult formulas containing any one of the oil blends of the invention.

Administration is usually via oral or enteral route, which may include the use of gavage feeding, with a gastric feeding tube, sonda, etc, particularly where adapted for preterm infant feeding, infant feeding or for clinical nutrition.

The lipid composition and/or vegetable-derived fat source according to the invention may be formulated as or into an edible product. To this end, the lipid composition and/or vegetable-derived fat source may be combined with at least one probiotic and prebiotic substance.

The edible product may be provided in fluid form (e.g. as a drink or beverage), as well as in a solid (e.g. as a powder) or semi solid form (e.g. as a porridge, or solid edible product).

In a further aspect, the invention relates to a commercial package for preparing an edible fat source or food article which is recommended for improving sleep and/or treating at least one sleep problem in a subject, in accordance with the invention. In addition to the active and non-active constituents, the commercial package contains instructions for use. These include terms of storage, instructions for preparation of the fat source or food article for administration, required dilutions, dosages, frequency of administration and the like. A commercial package in accordance with the invention may also contain the lipid composition and/or vegetable-derived fat source in a ready-to-use form, together with instructions for use. Dosages are usually determined according to age, weight, sex and condition of the subject, in accordance to good medical practice known to the attending physician and other medical personnel.

Thus, in yet a further one of its aspects, the present invention provides a commercial package comprising:

a) an edible lipid composition and/or vegetable-derived fat source as herein disclosed which upon enteral administration to a subject improves sleep in a subject and/or treats at least one sleep problem in a subject;
b) optionally, at least one of edible physiologically acceptable protein, carbohydrate, vitamin, mineral and active or non-active additives;
c) optionally, at least one edible physiologically acceptable carrier or diluent for carrying the constituent/s defined in a) and b);
d) means and receptacles for admixing the constituents defined in a), b) and/or c); and
e) instructions for use.

In some embodiments the lipid composition of the invention may be artificially enriched with at least one triglyceride. As used herein, the term “artificially enriched” is used to denote that the lipid composition, while typically originated from a natural lipid source, is subjected to at least one modification, typically an enzymatic processing step, albeit not limited thereto, that promotes enrichment of the lipids with at least one triglyceride as defined.

The natural lipid source may be any edible lipid source, preferably, a vegetable oil, including, without being limited thereto, soybean oil, high oleic sunflower oil, high oleic safflower oil palm oil, palm olein oil, canola oil, coconut oil, palm kernel oil, sunflower oil, corn oil, safflower and rapeseed oil.

The lipid composition and/or vegetable-derived fat source of the invention is preferably provided to the subject orally, e.g. as an edible product, as discussed herein.

The methods according to the invention may be short-term methods as well as long-term methods. In other words, the subject, in particular, the infant, toddler or child subject, may receive a single dose of the lipid composition (or fat source) or an edible product comprising the same, as well as a series of doses of the lipid composition (or fat source), per day, a series of doses along a period of several days, weeks, months and 1, 2, 3 or more years. It is appreciated that when the methods according to the invention are conducted for a long period of time, the composition and/or the fat source and/or the product may vary depending on the age of the subject, as well as other considerations such as nutritional needs. Administration may commence at any time from day one after birth. Administration may also be to a breastfed subject, as supplementary feedings, or during or after weaning, or when the breastfeeding person (usually mother) is absent or unable to breastfeed.

In some embodiments the triglyceride according to the invention is selected from the group consisting of naturally occurring triglycerides, synthetic triglycerides semi-synthetic triglycerides, and artificially produced triglycerides, all derived from a vegetable source.

As used herein, the forms “a”, “an” and “the” include singular as well as plural references unless the context clearly dictates otherwise. For example, the term “a triglyceride” includes one or more triglycerides which may form together a lipid base or a lipid blend.

The term “consisting essentially of” is used to define the lipid composition which include the recited elements but exclude other elements, i.e. the term lipid composition is used to define a composition consisting essentially only lipids. “Consisting of” shall thus mean excluding more than trace elements of other elements. Embodiments defined by each of these transition terms are within the scope of this invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Further, all numerical values, e.g. when referring the amounts or ranges of the elements constituting the various lipid compositions herein are approximations which are varied (+) or (−) by up to 20%, at times by up to 10% of the stated values. It is to be understood, even if not always explicitly stated that all numerical designations are preceded by the term “about”.

It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

It is noted that features of certain embodiments of the invention which are described in detail in the context of one aspect of the invention, may be applicable in other aspects of the invention.

The invention will now be exemplified in the following description of experiments that are carried out in accordance with the invention. It is to be understood that these examples are intended to be in the nature of illustration rather than of limitation. Obviously, many modifications and variations of these examples are possible in light of the above teaching.

It is therefore, to be understood that within the scope of the appended claims, the invention may be practiced otherwise, in a myriad of possible ways, than as specifically described herein below.

### DESCRIPTION OF NON-LIMITING EXAMPLES

In the present description as well as in the non-limiting examples provided below reference is made to fat bases and fat blends. It is to be understood that the term “fat base” or “fat concentrate” or “fat base concentrate” is used to denote the enzymatically prepared lipid composition comprising a mixture of vegetable-derived triglycerides with high sn-2 palmitic acid; while the term “fat blend” is used to denote a lipid composition comprising a fat base and a mixture of edible vegetable oil/s.

As shown below, the fat blend is a fat base comprising mainly triglycerides with high total palmitic and high sn-2 palmitic acid mixed with other edible vegetable oils. The edible vegetable oil/s may be natural vegetable oil/s, randomized vegetable oil/s, interesterified vegetable oils, enzymatically interesterified vegetable oils, at least two vegetable oils which were co-randomized, at least two vegetable oils which were co-interesterified. Generally, this fat blend is used as a fat fraction in infant formulas and can be used in other baby foods such as biscuits, bar, etc., food articles, clinical nutrition products or adults formula.

As used herein, the term “palmitic acid sn-2 ratio” or “C16:0 sn-2 ratio” means the level of palmitic acid moieties at the sn-2 position of the glycerol backbone as % of total palmitic acid in the triglyceride composition (oil).

As used herein, the term “TG” refers to triglyceride.

### Example 1—Preparing Fat Bases and Fat Blends

Table 1 details the contents of several fat bases enriched with a high content of palmitic acid at the sn-2 position. The fat bases comprise a high percentage of palmitic acid, C16:0, at the sn-2 position of triacylglycerol (TAG), and high percentage of unsaturated fatty acids at the sn-1 and sn-3 positions.

The fat bases are prepared as described in publication WO 2005/036987 which is fully incorporated herein by reference. Generally, a mixture of triglycerides, rich in palmitic acid (preferably above 78%) are reacted with a mixture of free fatty acids (FFA) rich in oleic acid (preferably above 75%), with a low content of palmitic and stearic acids (preferably below 6%).

Briefly, the triglyceride mixture may be produced from double-fractioned palm stearin and the FFA mixture is obtained from palm kernel oil after fractionation, or from high oleic sunflower oil. The two mixtures are blended in stirred (optionally large scale) reactors with no additional solvent. To this mixture is added a suitable lipase and the mixture of triglycerides, FFA and catalyst is stirred at 50° C.−60° C. for about 3-9 hours, to yield the final and desired triglycerides mixture. Any excess FFAs are removed.

The triglyceride product may be further treated in order to improve color, odor and taste with bleaching and deodorization stages. Optionally, the product is fortified with natural antioxidants to increase the shelf life of the product. The catalyst can be further recycled, to be re-used in further batches.

TABLE 1
Fat bases composition

| Fat Base No./ | | | | | | | | | | | |
|---|---|---|---|---|---|---|---|---|---|---|---|
| fatty acid* | 1 | 2 | 3 | 4 | 5 | 6 | 7 | 8 | 9 | 10 | 11 |
| C16:0 | 32 | 29.4 | 29.6 | 32.6 | 32.2 | 30.6 | 29 | 29 | 30 | 33 | 30 |
| C16:0 at sn-2 | 67.2 | 59.7 | 61.3 | 66.1 | 66 | 62.9 | 53.9 | 55.6 | 59 | 52.9 | 55.8 |
| total fatty acids | | | | | | | | | | | |
| at sn-2 | | | | | | | | | | | |
| Ratio (%) of | 70.0 | 67.7 | 69.0 | 67.6 | 68.3 | 68.5 | 62 | 64 | 64 | 53.5 | 62 |
| C16:0 at sn-2 | | | | | | | | | | | |
| out of total | | | | | | | | | | | |
| C16:0 | | | | | | | | | | | |
| C18:0 | 4 | 4.4 | 4.4 | 4 | 4.1 | 3.8 | 2.6 | 2.6 | 3 | 3 | 3 |
| C18:1 | 53.1 | 55.9 | 55.5 | 53.1 | 53.4 | 55 | 55.5 | 56 | 56.1 | 52 | 56.1 |
| C18:2 | 8 | 7.8 | 8.2 | 8 | 7.9 | 8.3 | 9 | 9 | 8.5 | 10 | 8.5 |

*All numbers represent % (w/w), meaning the weight % of the specific fatty acid out of total fatty acids weight, except for the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

The fat bases are then used to form the fat blends which comprise also other oils. The fat base may represent from about 25% up to about 83% of the fat blends suitable for use in a formula for use in the invention. The blends comprising the fat bases of Table 1 in combination with other fats are provided in Table 2.

Specifically, Table 2 details the contents of blends comprising one of fat bases 1, 7, 8, 9, 10 or 11. The fat blends are prepared by blending the selected fat base with other oils (e.g., palm kernel oil, coconut oil, palm oil, sunflower oil, corn oil, safflower oil, rapeseed oil and soybean oil). As such, the fatty acids composition of the blends results from the fatty acids composition of both the fat base and of the other oils mixed with the fat base.

TABLE 2
Fat blends composition

| | Fat | Fat | Fat | Fat | Fat | Fat | Fat | Fat | Fat | Fat |
|---|---|---|---|---|---|---|---|---|---|---|
| Fat Blend | blend | blend | blend | blend | blend | blend | blend | blend | blend | blend |
| No./fat* | 1 | 2 | 3 | 4 | 5 | 6 | 7 | 8 | 9 | 10 |
| C12:0 | 11.1 | 7.2 | 7.8 | 6.5 | 4.4 | 8.14 | 8.7 | 13.4 | 10.4 | 10 |
| C14:0 | 4.5 | 3.1 | 3.3 | 2.8 | 2.1 | 2.94 | 3.54 | 5.3 | 4.3 | 4.2 |
| C16:0 | 22.8 | 25.4 | 26.9 | 25.1 | 27.7 | 21.60 | 20.99 | 15 | 22.3 | 17 |
| C16:0 at s | 33.4 | 42.9 | 48.9 | 50.8 | 56.9 | 31.3 | 31.8 | 25 | 28.8 | 16 |
| n-2 of | | | | | | | | | | |
| total fatty | | | | | | | | | | |
| acids at sn-2 | | | | | | | | | | |
| Ratio (%) | 48.7 | 56.3 | 60.7 | 67.4 | 68.5 | 48.31 | 50.46 | 55 | 43 | 31.5 |
| sn-2 C16:0 of | | | | | | | | | | |
| total C16:0 | | | | | | | | | | |
| C18:0 | 2.3 | 3.0 | 3.1 | 3.5 | 4.0 | 2.65 | 2.65 | 2.9 | 4.4 | 3.2 |
| C18:1 | 38.4 | 40.8 | 41.6 | 47.9 | 46.6 | 42.71 | 44.37 | 39.7 | 38.5 | 41.7 |
| C18:2 | 13.5 | 15.6 | 12.8 | 8.6 | 11.7 | 17.96 | 16.43 | 15.3 | 14.0 | 18.2 |
| C18:3 | 1.7 | 0.6 | | 1.4 | | 1.69 | 1.52 | 2 | 1.5 | 2.1 |

*All numbers represent % (w/w), meaning the weight % of the specific fatty acid out of total fatty acids weight, except for: the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

Further blends are detailed in Tables 3 to 6 below.

Table 3 details the content of specific Fat blend 11.

TABLE 3
Fat blend 11 composition (with 30% fat base)

| Fatty acid | % of fatty acids* |
|---|---|
| C10:0 | 1.3 |
| C12:0 | 10.3 |
| C14:0 | 4.3 |
| C16:0 | 23.5 |
| C16:0 at sn-2 of total fatty acids | 30.3 |
| at sn-2 | |
| Ratio (%) of C16:0 at sn-2 of total | 43 |
| C16:0 | |
| C18:0 | 3.2 |
| C18:1 | 39.2 |
| C18:2 | 13.6 |
| C18:3 | 1.7 |
| C20:0 | 0.3 |
| C20:1 | 0.3 |
| C22:0 | 0.2 |
| % fat base in fat blend 11 | 30 |

*All numbers represent % (w/w), meaning the weight % of the specific fatty acid out of total fatty acids weight, except for: the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

Table 4 details the content of specific Fat blend 12.

TABLE 4
Fat Blend 12 composition (with 43% fat base)

| | % from total |
|---|---|
| Fatty acid | Fatty acids* |
| C8:0 | 1.6 |
| C10:0 | 1.5 |
| C12:0 | 10.6 |
| C14:0 | 3.9 |
| C16:0 | 17.2 |
| C16:0 at sn-2 of total fatty acids at sn-2 | 26.3 |
| Ratio (%) of sn-2 palmitic acid of total | 51 |
| palmitic acid | |
| C18:0 | 2.4 |
| C18:1 | 41.1 |
| C18:2 | 18.2 |
| C18:3 | 2.2 |
| % fat base (concentrate) in fat blend | 43 |
| Vegetable Oil | |
| Randomized Coconut oil | 22 |
| Randomized Sunflower | 15 |
| Randomized Rapeseed | 20 |

*All numbers represent % (w/w), meaning the weight % of the specific fatty acid out of total fatty acids weight, except for: the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

Table 5 details the content of specific Fat blend 13.

TABLE 5
Fat Blend 13 composition

| | | % (w/w) | | | % (w/w) |
|---|---|---|---|---|---|
| | | on | | | on |
| | % (w/w) | sn-2 | sn-2* | sn-⅓** | sn-⅓ |
| | on TG | position | ratio | ratio | position |
| C12 | 0.2 | 0.3 | 50.0 | 50.0 | 0.2 |
| C14 | 15.0 | 21.0 | 46.7 | 53.3 | 12.0 |
| C16 | 17.0 | 23.0 | 45.1 | 54.9 | 14.0 |
| C18 | 13.0 | 24.0 | 61.5 | 38.5 | 7.5 |
| C18:1n9 | 47.0 | 30.0 | 21.3 | 78.7 | 55.5 |
| C18:2n6 | 7.4 | 1.0 | 4.5 | 95.5 | 10.6 |
| C18:3n3 | 0.2 | 0.2 | 33.3 | 66.7 | 0.2 |
| saturated | 45.2 | 68.3 | | | 33.7 |
| unsaturated | 54.6 | 31.2 | | | 66.3 |
| palmitic of | 37.6 | 33.7 | | | 41.6 |
| saturated | | | | | |
| oleic of | 86.1 | 96.2 | | | 83.7 |
| unsaturated | | | | | |
| linoleic of | 13.6 | 3.2 | | | 16.0 |
| unsaturated | | | | | |
| linolenic of | 0.4 | 0.6 | | | 0.3 |
| unsaturated | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-⅓ ratio represents the ratio between the amount of certain fatty acid moiety at the sn-1 and sn-3 positions of the glycerol backbone out of total amount of said fatty acid in the triglyceride composition.
% (w/w) on sn-⅓ position represents the % of the specific fatty acid at sn-1 and sn-3 positions of the glycerol backbone out of total fatty acids at sn-1 and sn-3 positions of the glycerol backbone. For example, C16:0 sn2 ratio is calculated by: [(% of C16:0 at sn-2 out of total sn-2 positioned fatty acids)/3)/(% total C16:0)] × 100.

Table 6 details the content of specific Fat blend 14.

TABLE 6
Fat Blend 14 composition

| | | | | | % (w/w) |
|---|---|---|---|---|---|
| | % | % (w/w) | | | on |
| | (w/w) | on sn-2 | sn-2* | sn-⅓** | sn-⅓ |
| | on TG | position | ratio | ratio | position |
| C12 | 0.2 | 0.3 | 50.0 | 50.0 | 0.2 |
| C14 | 15.0 | 30.0 | 66.7 | 33.3 | 7.5 |
| C16 | 20.0 | 30.0 | 50.0 | 50.0 | 15.0 |
| C18 | 5.9 | 3.9 | 22.0 | 78.0 | 6.9 |
| C18:1n9 | 47.1 | 24.2 | 17.1 | 82.9 | 58.6 |
| C18:2n6 | 7.4 | 4.9 | 22.1 | 77.9 | 8.7 |
| C18:3n3 | 0.2 | 0.2 | 33.3 | 66.7 | 0.2 |
| saturated | 41.1 | 64.2 | | | 29.6 |
| unsaturated | 54.7 | 29.3 | | | 67.4 |
| palmitic of | 48.7 | 46.7 | | | 50.8 |
| saturated | | | | | |
| oleic of | 86.1 | 82.6 | | | 86.9 |
| unsaturated | | | | | |
| linoleic of | 13.5 | 16.7 | | | 12.8 |
| unsaturated | | | | | |
| linolenic of | 0.4 | 0.7 | | | 0.3 |
| unsaturated | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-⅓ ratio represents the ratio between the amount of certain fatty acid moiety at the sn-1 and sn-3 positions of the glycerol backbone out of total amount of said fatty acid in the triglyceride composition.
% (w/w) on sn-⅓ position represents the % of the specific fatty acid at sn-⅓ positions of the glycerol backbone out of total fatty acids at sn-1 and sn-3 positions of the glycerol backbone.
For example, C16:0 sn2 ratio is calculated by: [(% of C16:0 at sn-2 out of total sn-2 positioned fatty acids)/3)/(% total C16:0)] × 100.

### Example 2—the Effect of Infant Formula with Different Fat Components on Sleep in Formula-Fed Infants

The effect of the fat component in the infant formula on infant sleep was examined in a double blind randomized clinical trial in human term formula fed infants.

### Study Design

Healthy term infants (born at GA237) appropriate for gestational age and younger than 14 days were eligible for entry to this multi-center, randomized, double-blind study. Infants were excluded from the study if they suffered from a congenital or chromosomal disorder, neonatal morbidities, or metabolic illnesses.

Infants were randomly assigned to one of the two groups:

Group A: Infants fed with infant formula A (see below) containing a lipid composition according to the invention.
Group B: Infants fed with a control formula B (see below).

Both formulas were produced by the same manufacturer and under the same conditions, using identical ingredients from the same batches but the fat ingredient, which differed mainly in the fatty acid structural distribution.

The study was conducted according to the principles of the Declaration of Helsinki and good clinical practices. The protocol was approved by the Ethics Committees of clinical centers; all the parents gave written informed consent prior to inclusion.

Table 7 below details the composition of Formula A (according to the invention) and Formula B (control).

TABLE 7
Composition of Study Formulas (per 100 gr powder)

| | Formula A (per 100 gr | Formula B (per 100 gr |
|---|---|---|
| | powder) | powder) |
| Energy (kcal) | 502 | 498 |
| Protein (g) | 11.1 | 10.9 |
| Carbohydrate | 56.8 | 57.8 |
| (g) | | |
| Fat (g) | 25 | 24.5 |

Table 8 below details the fatty acids composition of Formula A (according to the invention) and Formula B (control).

TABLE 8
Fatty acids Composition of Study Formulas

| | Lipid composition of Formula A | | | | | Lipid composition of Formula B | | | | |
|---|---|---|---|---|---|---|---|---|---|---|
| | w/w | w/w | | | w/w | | w/w | | | w/w |
| | % | % on | | Sn- | % on | w/w | % on | | Sn- | % on |
| Sample | on | sn-2 | Sn-2* | 1/3** | sn-1/3 | % on | sn-2 | Sn-2* | 1/3** | sn-1/3 |
| name | TG | position | ratio | ratio | position | TG | position | ratio | ratio | position |
| C8 | 0.7 | — | — | — | — | 0.7 | — | — | — | — |
| C10 | 0.7 | 0.4 | — | — | — | 0.7 | 0.2 | — | — | — |
| C12 | 9.1 | 11.8 | 43.2 | 56.8 | 7.8 | 9.4 | 11.7 | 41.5 | 58.5 | 8.3 |
| C14 | 3.3 | 4 | 40.4 | 59.6 | 3.0 | 3.5 | 4.2 | 40.0 | 60.0 | 3.2 |
| C16 | 20.3 | 27 | 44.3 | 55.7 | 17.0 | 20.8 | 8.2 | 13.1 | 86.9 | 27.1 |
| C18 | 3.6 | 1.5 | 13.9 | 86.1 | 4.7 | 3.3 | 0.7 | 7.1 | 92.9 | 4.6 |
| C18:1 | 42.8 | 32.4 | 25.2 | 74.8 | 48.0 | 42.3 | 48.8 | 38.5 | 61.5 | 39.1 |
| C18:2 | 16.2 | 19.5 | 40.1 | 59.9 | 14.6 | 16 | 23.2 | 48.3 | 51.7 | 12.4 |
| C18:3 | 1.9 | 3.1 | 54.4 | 45.6 | 1.3 | 1.7 | 2.9 | 56.9 | 43.1 | 1.1 |
| Saturated | 37.7 | 44.7 | 39.5 | 60.5 | 32.3 | 38.4 | 25 | 21.7 | 78.3 | 43.1 |
| Unsaturated | 60.9 | 55 | 30.1 | 69.9 | 63.9 | 60 | 74.9 | 41.6 | 58.4 | 52.6 |
| Palmitic | 53.8 | 60.4 | | | 52.5 | 54.2 | 32.8 | | | 62.9 |
| of | | | | | | | | | | |
| saturated | | | | | | | | | | |
| oleic of | 70.3 | 58.9 | | | 75.2 | 70.5 | 65.2 | | | 74.3 |
| unsaturated | | | | | | | | | | |
| linoleic | 26.6 | 35.5 | | | 22.8 | 26.7 | 31.0 | | | 23.6 |
| of | | | | | | | | | | |
| unsaturated | | | | | | | | | | |
| Linolenic | 3.1 | 5.6 | | | 2.0 | 2.8 | 3.9 | | | 2.1 |
| unsaturated | | | | | | | | | | |

% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-1/3 ratio represents the ratio between the amount of certain fatty acid moiety at the sn-1 and sn-3 positions of the glycerol backbone out of total amount of said fatty acid in the triglyceride composition.
% (w/w) on sn-1/3 position represents the % of the specific fatty acid at sn-1/3 positions of the glycerol backbone out of total fatty acids at sn-1 and sn-3 positions of the glycerol backbone. For example, C16:0 sn2 ratio is calculated by: [(% of C16:0 at sn-2 out of total sn-2 positioned fatty acids)/3)/(% total C16:0)] × 100.

Parents' Questionnaires

Data on feeding and sleeping were reported by the parents, who used 24-hr behavior diaries for 3 days at age of 6, 12 and 24 weeks postnatal. The parents reported the duration and time of the day for each sleeping episode. Mean number of sleep periods per day, mean sleep duration per day, and the pattern of sleep during 24 hrs of day and night and during night hours only (20:00-06:00) were calculated and analyzed.

### Results

### Sleep Characteristics

As shown in Table 9, group A demonstrated an increase in sleep duration in comparison with group B at weeks 6 and 12 weeks. Additionally a slight increase in sleep duration during night hours was observed at 6 weeks among group A infants in comparison with group B.

Repeated measures statistical analysis revealed a significant difference in sleep pattern between the groups at 6 and 12 weeks.

TABLE 9
Mean sleep duration in groups A and B

| | Group A | Group B |
|---|---|---|
| Sleep duration at 6 weeks (hours) | 15.4 ± 2.7 | 14.3 ± 2.5 |
| Sleep duration at 12 weeks (hours) | 14.3 ± 2.7 | 13.7 ± 2.4 |
| Sleep duration during night (20:00-06:00) at | 7.0 ± 1.7 | 6.5 ± 1.6 |
| 6 weeks (hours) | | |
| Sleep duration during night (20:00-06:00) at | 7.2 ± 1.8 | 6.9 ± 1.7 |
| 12 weeks (hours) | | |

A subgroup analysis was performed on infants with sleep problems whose sleep duration per day was equivalent for less than 25^{th} percentile of sleep duration at 6 weeks (equivalent to 12.9 hrs, calculated based on data from all infants in the study). The results of the subgroup analysis are presented in Table 10.

At weeks 12 and 24 sleep duration of infants with sleep disorders in group A was longer as compared to the group B. Similar results were obtained when measuring sleep duration during night hours (20:00-06:00).

TABLE 10
Mean sleep duration in
groups A and B among infants with sleep problems

| | Group A | Group B |
|---|---|---|
| Sleep duration at 6 weeks (hours) | 11.70 | 11.51 |
| Sleep duration at 12 weeks (hours) | 13.85 | 11.77 |
| Sleep duration at 24 weeks (hours) | 13.66 | 12.50 |
| Sleep duration during night (20:00-06:00) at | 5.59 | 5.60 |
| 6 weeks (hours) | | |
| Sleep duration during night (20:00-06:00) at | 7.21 | 6.47 |
| 12 weeks (hours) | | |
| Sleep duration during night (20:00-06:00) at | 7.99 | 7.12 |
| 24 weeks (hours) | | |

### CONCLUSION

Infant formulas comprising a composition according to the invention had a positive effect on sleep patterns. The effect was even stronger among infants with sleep problems.

### Example 3—the Effect of Preterm Infant Formula with Different Fat Components on Sleep in Formula-Fed Preterm Infants

The effect of the fat component in the infant formula on infant sleep is examined in a double blind randomized clinical trial in human formula fed preterm infants.

### Study Design

Preterm infants (born at GA<37) that can be fed with enteral formula feeding are eligible for entry to this multi-center, randomized, double-blind study. Infants are excluded from the study if they suffer from a significant illness, a congenital or chromosomal disorder, neonatal morbidities or metabolic illnesses.

Infants are randomly assigned to one of the two groups:

Group A: Infants fed with infant formula A containing a lipid composition according to the invention (20% total palmitic acid, 48% of the palmitic acid at sn-2 position).
Group B: Infants fed with a control formula B (20% total palmitic acid, 14% of the palmitic acid at sn-2 position).

Formulas are produced by the same manufacturer and under the same conditions, using identical ingredients from the same batches but the fat ingredient.

The study is conducted according to the principles of the Declaration of Helsinki and good clinical practices. The protocol is approved by the Ethics Committees of clinical centers; all the parents give written informed consent prior to inclusion.

Table 11 below details the fatty acids composition of preterm Formula A (according to the invention) and Formula B (control).

TABLE 11
Fatty acids Composition of Study Formulas (%)

| | Lipid composition of Formula A | | | | | Lipid composition of Formula B | | | | |
|---|---|---|---|---|---|---|---|---|---|---|
| | w/w | w/w | | | w/w | | w/w | | | w/w |
| | % | % on | | Sn- | % on | w/w | % on | | Sn- | % on |
| Sample | on | sn-2 | Sn-2* | 1/3** | sn-1/3 | % on | sn-2 | Sn-2* | 1/3** | sn-1/3 |
| name | TG | position | ratio | ratio | position | TG | position | ratio | ratio | position |
| C8 | 0.7 | — | — | — | — | 0.7 | — | — | — | — |
| C10 | 0.7 | 0.4 | — | — | — | 0.7 | 0.2 | — | — | — |
| C12 | 13.8 | 11.8 | 43.2 | 56.8 | 7.8 | 13.6 | 11.7 | 41.5 | 58.5 | 8.3 |
| C14 | 6.3 | 2.1 | 21.2 | 78.8 | 3.9 | 6.4 | 4.2 | 40 | 60 | 3.2 |
| C16 | 15.4 | 29.3 | 48.1 | 51.9 | 15.8 | 15.3 | 8.8 | 14.1 | 85.9 | 26.8 |
| C18 | 3.6 | 1.5 | 13.9 | 86.1 | 4.7 | 3.7 | 0.7 | 7.1 | 92.9 | 4.6 |
| C18:1 | 43.1 | 32.1 | 25 | 75 | 48.2 | 43.2 | 46.6 | 36.7 | 63.3 | 40.2 |
| C18:2 | 14.3 | 19.5 | 40.1 | 59.9 | 14.6 | 14.4 | 23.2 | 48.3 | 51.7 | 12.4 |
| C18:3 | 1.9 | 3.1 | 54.4 | 45.6 | 1.3 | 1.8 | 2.9 | 56.9 | 43.1 | 1.1 |
| Saturated | 40.5 | 45.1 | 37.1 | 62.9 | 32.2 | 40.4 | 25.6 | 21.1 | 78.9 | 42.9 |
| Unsaturated | 59.3 | 54.7 | 30.7 | 69.3 | 64.1 | 59.4 | 72.7 | 40.8 | 59.2 | 53.7 |
| Palmitic | 38.0 | 65.0 | | | 49.1 | 37.9 | 34.4 | | | 62.5 |
| of | | | | | | | | | | |
| saturated | | | | | | | | | | |
| oleic of | 72.7 | 58.7 | | | 75.2 | 72.7 | 64.1 | | | 74.9 |
| unsaturated | | | | | | | | | | |
| linoleic | 24.1 | 35.6 | | | 22.8 | 24.2 | 31.9 | | | 23.1 |
| of | | | | | | | | | | |
| unsaturated | | | | | | | | | | |
| Linolenic | 3.2 | 5.7 | | | 2.0 | 3.0 | 4.0 | | | 2.0 |
| of | | | | | | | | | | |
| unsaturated | | | | | | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-1/3 ratio represents the ratio between the amount of certain fatty acid moiety at the sn-1 and sn-3 positions of the glycerol backbone out of total amount of said fatty acid in the triglyceride composition.
% (w/w) on sn-1/3 position represents the % of the specific fatty acid at sn-1/3 positions of the glycerol backbone out of total fatty acids at sn-1 and sn-3 positions of the glycerol backbone.
For example, C16:0 sn2 ratio is calculated by: [(% of C16:0 at sn-2 out of total sn-2 positioned fatty acids)/3)/(% total C16:0)] × 100.

### Parents' Questionnaires

Data on feeding and sleeping are reported by the parents or medical staff, who use 24-hr behavior diaries for 3 days at age of 6, 12 and 24 weeks postnatal. The report includes the duration and time of the day for each sleeping episode. Mean number of sleep periods per day, mean sleep duration per day, and the pattern of sleep during 24 hrs of day and night and during night hours only (20:00-06:00) are calculated and analyzed.

### Results

### Sleep Characteristics

Group A demonstrates an increase in sleep duration in comparison with group B at weeks 6 and 12 weeks.

### CONCLUSION

Infant formulas comprising a composition according to the invention has a positive effect on sleep patterns of preterm infants.

1. An edible lipid composition comprising a vegetable-derived fat source, wherein said fat source is a triglyceride fat source comprising triglycerides with 15-55% palmitic acid moieties out of the total fatty acids, and wherein the level of palmitic acid moieties at the sn-2 position of the glycerol backbone is at least 30% of total palmitic acid, for use in a method for one or both of improving sleep in a subject and treating at least one sleep problem in a subject.

2. The composition according to claim 1 wherein at least about 20% of the total fatty acid moieties at the sn-2 position of the glycerol backbone are saturated.

3. The composition according to claim 1 or 2 wherein at least about 45% of the total fatty acid moieties at the sn-1 and sn-3 positions of the glycerol backbone are unsaturated.

4. The composition according to any one of claims 1 to 3 wherein at least about 40% of the fatty acids are unsaturated fatty acid moieties.

5. The composition according to any one of claims 1 to 4 wherein at least about 50% of the unsaturated fatty acids are oleic acid.

6. The composition according to of any one of claims 1 to 5 wherein at least about 10% of the unsaturated fatty acids are linoleic acid.

7. The composition according to any one of claims 1 to 6 wherein at least about 1% of the unsaturated fatty acids are linolenic acid.

8. The composition according to any one of claims 1 to 7 wherein at least about 35% of the unsaturated fatty acid moieties at the sn-1 and sn-3 positions are oleic acid moieties.

9. The composition according to any one of claims 1 to 8 wherein at least about 4% of the unsaturated fatty acid moieties at the sn-1 and sn-3 positions are linoleic acid moieties.

10. The composition according to any one of claims 1 to 9 wherein said subject is an adult.

11. The composition according to any one of claims 1 to 9 wherein said subject is an infant.

12. The composition according to any one of claims 1 to 9 wherein said subject is a preterm infant.

13. The composition according to any one of claims 1 to 12, wherein said subject is a healthy subject.

14. The composition according to any one of claims 1 to 12, wherein said subject is a non-healthy subject.

15. The composition according to any one of claims 1 to 14, wherein said method is for improving sleep.

16. The composition according to any one of claims 1 to 15, wherein said method is for treating at least one sleep problem.

17. The composition according to any one of claims 1 to 16, wherein said method is for increasing sleep duration.

18. The composition according to claim 17, wherein said method is for increasing night sleep duration.

19. The composition according to any one of claims 1 to 18 wherein said subject is suffering from a sleep problem.

20. The composition according to claim 19, wherein said sleep problem is insufficient sleeping duration.

21. The composition according to claim 20, wherein said sleep problem is insufficient night sleep duration.

22. The composition according to any one of claims 1 to 21 wherein said sleep problem is reduced sleeping hours.

23. A formula comprising the composition according to any one of claims 1 to 22 for use in a method for one or both of improving sleep in a subject and treating at least one sleep problem in a subject.

24. The formula of claim 23, being an infant formula.

25. The formula of claim 23, being an adult formula.

26. An edible vegetable-derived fat source, wherein said fat source is a triglyceride fat source comprising triglycerides with 15-55% palmitic acid moieties out of the total fatty acids and wherein the level of palmitic acid moieties at the sn-2 position of the glycerol backbone is at least 30% of total palmitic acid, for use in a method for one or both of improving sleep in a subject and treating at least one sleep problem in a subject.

27. A formula comprising the composition according to any one of claims 1 to 22 or the fat source according to claim 26, for use in a method for one or both of improving sleep in a subject and treating at least one sleep problem in a subject.

28. The formula of claim 27, being a preterm infant formula.

29. The formula of claim 27, being an infant formula.

30. The formula of claim 27, being a toddler formula.

31. The formula of claim 27, being a child formula.

32. The formula of claim 27, being an adolescent formula.

33. The formula of claim 27, being an adult formula.

34. The formula of claim 27, being geriatric formula.

35. A commercial package comprising:
a) the composition according any one of claims 1 to 22 or the fat source according to claim 26 which upon enteral administration to a subject improves sleep in said subject and/or treats at least one sleep problem in said subject;
b) optionally, at least one of edible physiologically acceptable protein, carbohydrate, vitamin, mineral and active or non-active additives;
c) optionally, at least one edible physiologically acceptable carrier or diluent for carrying the constituent/s defined in a) and b);
d) means and receptacles for ad mixing the constituents defined in a), b) and/or c); and
e) instructions for use.
